(19) United States
(12) Patent Application Publication
(10) Pub. No.: US 2025/0074883 A1
(43) Pub. Date: Mar. 6, 2025

(54) 3-NITRO-1,2,4-TRIAZOLE-5-ONE-D2, PREPARATION METHOD AND USE THEREOF

(71) Applicant: Beijing Inst of Tech Deuterium Material Co., Ltd., Beijing (CN)

(72) Inventors: Jiping LIU, Beijing (CN); Zhuqing FANG, Beijing (CN); Jia HAN, Beijing (CN)

(21) Appl. No.: 18/242,865

(22) Filed: Sep. 6, 2023

(51) Int. Cl. C07D 249/14; C06B 25/34

(52) U.S. Cl. CPC C07D 249/14; C06B 25/34; C07B 2200/05; C07B 2200/13

(57) ABSTRACT

Provided are 3-nitro-1,2,4-triazole-5-one-d2 and a preparation method and use thereof. Compared with 3-nitro-1,2,4-triazole-5-one, the 3-nitro-1,2,4-triazole-5-one-d2 provided by the disclosure has an increased molecular relative molar mass, which leads to the increase in crystal density. By substituting hydrogen atoms with deuterium atoms, it is possible to avoid the influence of hydrogen atoms on neutron diffraction signal-to-noise ratio. Compared with carbon-hydrogen bond, carbon-deuterium bond has lower zero point energy, lower vibration frequency and more stability, allowing 3-nitro-1,2,4-triazole-5-one-d2 to have high density, excellent detonation performance, high detonation velocity, high stability, low thermal sensitivity, low friction sensitivity and low impact sensitivity.

### TECHNICAL FIELD

The present disclosure relates to the technical field of deuterated energetic materials, in particular to 3-nitro-1,2,4-triazole-5-one-d2 and a preparation method thereof.

### BACKGROUND

3-nitro-1,2,4-triazole-5-one is a typical low-sensitive high explosive, with a density as high as 1.93 g/cm³. It has an explosion capacity similar to that of hexogeon (RDX), but is much more insensitive than RDX. Due to excellent performance and low cost, 3-nitro-1,2,4-triazole-5-one is often used in melt-cast explosives, cast explosives and pressed explosives. However, 3-nitro-1,2,4-triazole-5-one has a low detonation velocity, and the detonation performance thereof needs to be improved.

### SUMMARY

In view of the above, an object of the present disclosure is to provide 3-nitro-1,2,4-triazole-5-one-d2 and a preparation method and use thereof. The 3-nitro-1,2,4-triazole-5-one-d2 provided by the present disclosure has a high detonation velocity and excellent detonation performance.

In order to achieve the above object, the present disclosure provides the following technical solutions.

The present disclosure provides 3-nitro-1,2,4-triazole-5-one-d2, having a structure represented by formula I:

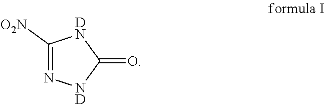

The present disclosure provide a method for preparing the 3-nitro-1,2,4-triazole-5-one-d2 of the above technical solution, comprising following steps:
mixing deuterated semicarbazide hydrochloride and a deuterated formic acid aqueous solution to obtain a first mixed solution, subjecting the first mixed solution to condensation reaction to obtain a crude 1,2,4-triazole-5-one-d3, and subjecting the crude 1,2,4-triazole-5-one-d3 to recrystallization to obtain 1,2,4-triazole-5-one-d3, and
mixing the 1,2,4-triazole-5-one-d3, a deuterated nitric acid aqueous solution, phosphoric anhydride and a chloride at a temperature of not greater than 5° C. to obtain a second mixed solution, and subjecting the second mixed solution to nitration reaction to obtain the 3-nitro-1,2,4-triazole-5-one-d2.

In some embodiments, the deuterated formic acid aqueous solution has a mass concentration of 60-90%, and
a molar ratio of the deuterated semicarbazide hydrochloride to deuterated formic acid in the deuterated formic acid aqueous solution is in a range of 1:(2-6).

In some embodiments, the condensation reaction comprises first condensation reaction and second condensation reaction which are sequentially performed, the first condensation reaction is performed at a temperature of 90-95° C. for 2-3 h, and the second condensation reaction is performed at a temperature of 100-120° C. for 4-6 h in a protective atmosphere.

In some embodiments, a recrystallization solvent used in the recrystallization comprises one or more selected from the group consisting of water and an alcohol; the alcohol comprises one or more selected from the group consisting of methanol, ethanol, ethylene glycol, propanol and glycerol, and
a mass ratio of the crude 1,2,4-triazole-5-one-d3 to the recrystallization solvent is in a range of 1:(2-5).

In some embodiments, the recrystallization comprises thermal dissolution, first cooling crystallization and second cooling crystallization which are sequentially performed,
the thermal dissolution is performed at a temperature of 50-100° C.,
the first cooling crystallization has a final temperature of 10-20° C., and is performed at a cooling rate of 1-10° C./min, and
the second cooling crystallization has a final temperature of not greater than 5° C., and is performed at a cooling rate of 0.5-1° C./min.

In some embodiments, mixing the 1,2,4-triazole-5-one-d3, the deuterated nitric acid aqueous solution, phosphoric anhydride and the chloride is performed as follows: dropwise adding the deuterated nitric acid aqueous solution into the 1,2,4-triazole-5-one-d3, then sequentially adding phosphoric anhydride and the chloride, and mixing,
the 1,2,4-triazole-5-one-d3 has a temperature of −5° C. to 0° C., and
the deuterated nitric acid aqueous solution is dropwise added at a rate of 0.5-3 mL/min.

In some embodiments, the deuterated nitric acid aqueous solution has a mass concentration of 90-95%,
a molar ratio of the 1,2,4-triazole-5-one-d3 to deuterated nitric acid in the deuterated nitric acid aqueous solution is in a range of 1:(8-12),
a molar ratio of phosphoric anhydride to deuterated nitric acid in the deuterated nitric acid aqueous solution is in a range of 1:(5-10),
a mass ratio of the 1,2,4-triazole-5-one-d3 to the chloride is in a range of (5-10): 1, and
the chloride comprises one or more selected from the group consisting of boron trichloride, aluminum trichloride, silicon tetrachloride and ferric trichloride.

In some embodiments, the nitration reaction is performed at a temperature of 60-80° C. for 4-8 h.

The present disclosure provides use of the 3-nitro-1,2,4-triazole-5-one-d2 described in the above technical solutions or the 3-nitro-1,2,4-triazole-5-one-d2 prepared by the method described in the above technical solutions in a national defense field.

The present disclosure provides 3-nitro-1,2,4-triazole-5-one-d2 having a structure represented by formula I. Compared with 3-nitro-1,2,4-triazole-5-one, the 3-nitro-1,2,4-triazole-5-one-d2 provided by the present disclosure has an increased molecular relative molar mass, which leads to an increased crystal density. In the present disclosure, melt-cast explosives, cast explosives, and pressed explosives prepared based on the 3-nitro-1,2,4-triazole-5-one-d2 have greater density, better detonation performance, and significantly improved detonation velocity. By substituting hydrogen atoms with deuterium atoms, it is possible to avoid the influence of hydrogen atoms in 3-nitro-1,2,4-triazole-5-one on neutron diffraction signal-to-noise ratio. Therefore, the 3-nitro-1,2,4-triazole-5-one-d2 provided by the present disclosure has better detonation performance and significantly improved detonation velocity. Compared with carbon-hydrogen bond, carbon-deuterium bond has a lower zero point energy, but their transition state activation energy is similar, which leads to a lower vibration frequency of carbon-deuterium bond than carbon-hydrogen bond, indicating that the 3-nitro-1,2,4-triazole-5-one-d2 is more stable. Therefore, compared with 3-nitro-1,2,4-triazole-5-one, the 3-nitro-1,2,4-triazole-5-one-d2 is more stable, and has a reduced friction sensitivity, a reduced thermal sensitivity, and a reduced impact sensitivity, thus broadening the application scope of the 3-nitro-1,2,4-triazole-5-one-d2. Moreover, by using the 3-nitro-1,2,4-triazole-5-one-d2, it is possible to study its thermal decomposition components more accurately, thus obtaining its possible thermal decomposition mechanism and decomposition kinetics. The 3-nitro-1,2,4-triazole-5-one-d2 provided by the present disclosure is of great application value and important research significance in the field of national defense as an explosive (especially a low-sensitive high explosive) or as a raw material for preparing explosives.

The present disclosure provides a method for preparing the 3-nitro-1,2,4-triazole-5-one-d2 described in the above technical solutions, which comprises the following steps: mixing deuterated semicarbazide hydrochloride and a deuterated formic acid aqueous solution to obtain a first mixed solution, subjecting the first mixed solution to condensation reaction to obtain a crude predeuterated 1,2,4-triazole-5-one-d3, and subjecting the crude 1,2,4-triazole-5-one-d3 to recrystallization to obtain 1,2,4-triazole-5-one-d3; and mixing the 1,2,4-triazole-5-one-d3, a deuterated nitric acid aqueous solution, phosphoric anhydride and a chloride at a temperature of not greater than 5° C. to obtain a second mixed solution, and subjecting the second mixed solution to nitration reaction to obtain the 3-nitro-1,2,4-triazole-5-one-d2. Compared with traditional methods for preparing 3-nitro-1,2,4-triazole-5-one (such as EP0585235A1; U.S. Ser. No. 06/583,293B1; Li Jiarong. One-pot Synthesis of 3-nitro-1,2,4-triazol-5-one [J]. Journal of Beijing Institute of Technology, 1998, 18 (4): 518-519; Mukundan T, Purandare G N, Nair J K, et al. Explosive Nitrotriazolone Formulates[J]. Defence Science Journal, 2002, 52(2): 127-133), the method provided by the present disclosure could greatly reduce the ring-opening decomposition of 1,2,4-triazole-5-one-d3 in the initial process of adding deuterated nitric acid by controlling the system temperature during mixing in the nitration reaction step to be not greater than 5° C., thereby improving the product yield. Moreover, compared with conventional concentrated sulfuric acid accelerant, in the present disclosure, phosphoric anhydride and a chloride are used as nitration accelerants, which reduces the rate of the nitration reaction, makes the nitration process more moderate, reduces the possibility of temperature runaway, solves the problem of large heat release in the reaction process, and makes it more easier to control the temperature of the nitration process, thus greatly reducing the potential safety hazard.

### DETAILED DESCRIPTION OF THE EMBODIMENTS

The present disclosure provides 3-nitro-1,2,4-triazole-5-one-d2, having a structure represented by formula I:

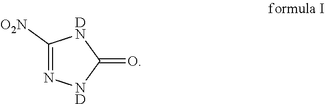

The present disclosure provides a method for preparing the 3-nitro-1,2,4-triazole-5-one-d2 as described above, comprising that following steps:
mixing deuterated semicarbazide hydrochloride and a deuterated formic acid aqueous solution to obtain a first mixed solution, subjecting the first mixed solution to condensation reaction to obtain a crude 1,2,4-triazole-5-one-d3, and subjecting the crude 1,2,4-triazole-5-one-d3 to recrystallization to obtain 1,2,4-triazole-5-one-d3; and
mixing the 1,2,4-triazole-5-one-d3, a deuterated nitric acid aqueous solution, phosphoric anhydride and a chloride at a temperature of not greater than 5° C. to obtain a second mixed solution, and subjecting the second mixed solution to nitration reaction to obtain the 3-nitro-1,2,4-triazole-5-one-d2.

In the present disclosure, unless otherwise specified, all raw material components are commercially available commodities well known to those skilled in the art.

In the present disclosure, the deuterated semicarbazide hydrochloride and the deuterated formic acid aqueous solution are mixed to obtain a first mixed solution, the first mixed solution is subjected to condensation reaction to obtain a crude 1,2,4-triazole-5-one-d3, and the crude 1,2,4-triazole-5-one-d3 is subjected to recrystallization to obtain 1,2,4-triazole-5-one-d3.

In some embodiments, the deuterated formic acid aqueous solution has a mass concentration of 60-90%, preferably 65-85%, and more preferably 70-80%. In some embodiments, a molar ratio of the deuterated semicarbazide hydrochloride to deuterated formic acid in the deuterated formic acid aqueous solution is in a range of 1:(2-6), preferably 1:(2.5-5.5), and more preferably 1:(3-5).

In the present disclosure, there is no special limitation on the mixing, and any mixing method well known to those skilled in the art, such as mixing by stirring, may be used, as long as the raw materials could be evenly mixed.

In some embodiments, the condensation reaction comprises first condensation reaction and second condensation reaction which are sequentially performed. In some embodiments, the first condensation reaction is performed at a temperature of 90-95° C., preferably 91-94° C., and more preferably 92-93° C. In some embodiments, the first condensation reaction is performed for 2-3 h, preferably 2.2-2.8 h, and more preferably 2.5 h. In some embodiments, the second condensation reaction is performed at a temperature of 100-120° C., preferably 105-115° C., and more preferably 110° C. In some embodiments, the second condensation reaction is performed for 4-6 h, preferably 4.5-5.5 h, and more preferably 5 h. In some embodiments, the second condensation reaction is performed in a protective atmosphere. In the present disclosure, there is no special limitation on the protective atmosphere, and any protective atmosphere well known to those skilled in the art, such as nitrogen, helium or argon, may be used. In some embodiments of the present disclosure, the condensation reaction comprises: placing the first mixed solution obtained by mixing in a pressure-resistant container, sealing the pressure-resistant container, subjecting the first mixed solution to first heating to a temperature of the first condensation reaction under stirring, and holding for the first condensation reaction, and then subjecting the first mixed solution obtained after the first condensation reaction to second heating to a temperature of the second condensation, and holding for the second condensation. In the present disclosure, there is no special limitation on the heating rates of the first heating and second heating, as long as the temperature could be raised to the temperature of the first condensation reaction and the second condensation reaction. In some embodiments, the stirring is performed at a rate of 50 to 100 rpm, preferably 70 to 80 rpm. In some embodiments, the pressure-resistant container is provided with a mechanical agitator, a constant-pressure dropping funnel and a condenser.

In some embodiments, after the condensation reaction is completed, the method further comprises cooling the obtained condensation reaction solution to ambient temperature, then subjecting the cooled condensation reaction solution to solid-liquid separation to obtain a solid product, and drying the solid product to obtain the crude 1,2,4-triazole-5-one-d3. In the present disclosure, there is no special limitation on the cooling, and any cooling means well known to those skilled in the art, such as natural cooling, may be used. In the present disclosure, there is no special limitation on the solid-liquid separation, and any solid-liquid separation means well known to those skilled in the art, such as suction filtration, may be used. In some embodiments, the drying is performed at a temperature of 20-60° C., preferably 30-50° C., and more preferably 40° C. In some embodiments, the drying is performed for 12-24 h, preferably 15-22 h, and more preferably 18-20 h. In some embodiments, the drying is vacuum drying.

In the present disclosure, after the crude 1,2,4-triazole-5-one-d3 is obtained, the crude 1,2,4-triazole-5-one-d3 is subjected to recrystallization to obtain 1,2,4-triazole-5-one-d3.

In some embodiments, a recrystallization solvent used in the recrystallization comprises one or more selected from the group consisting of water and an alcohol. In some embodiments, the water is deionized water. In some embodiments, the alcohol comprises one or more selected from the group consisting of methanol, ethanol, ethylene glycol, propanol and glycerol. In the present disclosure, there is no special limitation on the volume ratio of water to the alcohol, and any ratio can be used. In some embodiments, a mass ratio of the crude 1,2,4-triazole-5-one-d3 to the recrystallization solvent is in a range of 1:(2-5), preferably 1:(2.5-4.5), and more preferably 1:(3-4).

In the present disclosure, the recrystallization comprises thermal dissolution, first cooling crystallization and second cooling crystallization which are sequentially performed. In some embodiments, the thermal dissolution is performed at 50-100° C., preferably 60-90° C., and more preferably 70-80° C. In some embodiments, the first cooling crystallization has a final temperature of 10-20° C., preferably 12-18° C., and more preferably 14-15° C. In some embodiments, the first cooling crystallization is performed at a cooling rate of 1-10° C./min, preferably 2-8° C./min, and more preferably 3-7° C./min. In some embodiments, the second cooling crystallization has a final temperature of not greater than 5° C., preferably 0-5° C., and more preferably 1-4° C. In some embodiments, the second cooling crystallization is performed at a cooling temperature of 0.5-1° C./min, preferably 0.6-0.9° C./min, and more preferably 0.7-0.8° C./min.

In some embodiments, the recrystallization comprises: filling the pressure-resistant container with the recrystallization solvent, placing the pressure-resistant container in a high-precision medium-temperature circulating bath at a temperature of 20-25° C., adding the crude 1,2,4-triazole-5-one-d3 into the recrystallization solvent, sealing the pressure-resistant container, heating the resulting mixture to a temperature of 50-100° C., and stirring until the crude 1,2,4-triazole-5-one-d3 is dissolved, and then cooling to a temperature of 10-20° C. at a cooling rate of 1-10° C./min, and then cooling to a temperature of not greater than 5° C. at a cooling rate of 0.5-1° C./min. In some embodiments, the pressure-resistant container is provided with a mechanical agitator, a constant-pressure dropping funnel and a condenser.

In some embodiments, after the recrystallization, the method further comprises subjecting the obtained recrystallization solution to solid-liquid separation to obtain a solid product, washing the solid product with water and drying to obtain the 1,2,4-triazole-5-one-d3. In the present disclosure, there is no special limitation on the solid-liquid separation, and any solid-liquid separation method well known to those skilled in the art, such as suction filtration may be used. In some embodiments, water used in the washing has a temperature of not greater than 5° C., preferably 0-5° C., and more preferably 1-4° C. In some embodiments, the water used in washing is deionized water. In some embodiments, the washing is performed for 3-5 times, and preferably 4 times. In some embodiments, the drying is performed at a temperature of 20-40° C., preferably 25-35° C., and more preferably 40° C. In some embodiments, the drying is performed for 12-24 h, preferably 15-22 h, and more preferably 18-20 h. In some embodiments, the drying is vacuum drying.

In the present disclosure, after the 1,2,4-triazole-5-one-d3 is obtained, the 1,2,4-triazole-5-one-d3, a deuterated nitric acid aqueous solution, phosphoric anhydride and a chloride are mixed at a temperature of not greater than 5° C. to obtain a second mixed solution, and the second mixed solution is subjected to nitration reaction to obtain 3-nitro-1,2,4-triazole-5-one-d2.

In some embodiments, the deuterated nitric acid aqueous solution has a mass concentration of 90-95%, preferably 91-94%, and more preferably 92-93%. In some embodiments, a molar ratio of the 1,2,4-triazole-5-one-d3 to deuterated nitric acid is in a range of 1:(8-12), preferably 1:(9-11), and more preferably 1:10.

In some embodiments, a molar ratio of phosphoric anhydride to deuterated nitric acid is in a range of 1:(5-10), preferably 1:(6-9), and more preferably 1:(7-8).

In some embodiments, the chloride comprises one or more selected from the group consisting of boron trichloride, aluminum trichloride, silicon tetrachloride and ferric trichloride, and preferably comprises one selected from the group consisting of boron trichloride, aluminum trichloride, silicon tetrachloride or ferric trichloride. In some embodiments, a mass ratio of the 1,2,4-triazole-5-one-d3 to the chloride is in a range of (5-10): 1, preferably (6-9): 1, and more preferably (7-8): 1.

In some embodiments, the mixing is performed as follows: dropwise adding the deuterated nitric acid aqueous solution into the 1,2,4-triazole-5-one-d3, then sequentially adding phosphoric anhydride and the chloride, and mixing. In some embodiments of the present disclosure, the mixing is performed as follows: placing a pressure-resistant container filled with the 1,2,4-triazole-5-one-d3 in a high-precision medium-temperature circulating bath at a temperature of −5-0° C., dropwise adding the deuterated nitric acid aqueous solution into the 1,2,4-triazole-5-one-d3 while holding the system at a temperature of not greater than 5° C., and after the dropwise addition is completed, sequentially adding phosphoric anhydride and the chloride, and mixing. In some embodiments, the pressure-resistant container is provided with a mechanical agitator, a constant-pressure dropping funnel and a condenser. In some embodiments, the temperature of the high-precision medium-temperature circulating bath (that is, the temperature of the 1,2,4-triazole-5-one-d3) is in a range of −4 to −1° C., and preferably −3 to −2° C. In some embodiments, the dropwise adding is performed at a rate of 0.5-3 mL/min, preferably 1-2.5 mL/min, and more preferably 1.5-2 mL/min. In some embodiments, the dropwise adding is performed by using a constant pressure dropping funnel. By controlling the rate of the dropwise adding of the deuterated nitric acid aqueous solution, the system is controlled at a temperature not greater than 5° C. during the mixing, thus greatly reducing the ring-opening decomposition of the 1,2,4-triazole-5-one-d3 in the initial process of adding the deuterated nitric acid aqueous solution, and improving the product yield. The addition of the chloride promotes the reaction to nitration, thus inhibiting the ring-opening reaction.

In some embodiments, the nitration reaction is performed at a temperature of 60-80° C., preferably 65-75° C., and more preferably 70° C. In some embodiments, the nitration reaction is performed for 4-8 h, preferably 5-7 h, and more preferably 6 h. In some embodiments, the nitration reaction is performed under the conditions of stirring and sealing, and the stirring is performed at a rate of 300-500 rpm, preferably 400 rpm.

In some embodiments, after the nitration reaction is completed, the method further comprises subjecting the obtained nitration reaction solution to purification, wherein the purification comprises the following steps: sequentially subjecting the nitration reaction solution to cooling crystallization and solid-liquid separation to obtain a solid product, washing the solid product with water, and drying to obtain the 3-nitro-1,2,4-triazole-5-one-d2. In some embodiments, the cooling crystallization has a final temperature of not greater than 10° C., preferably 0-5° C. In some embodiments, the cooling crystallization is performed at a cooling rate of 1-10° C./min, preferably 2-8° C./min, and more preferably 3-7° C./min. In the present disclosure, there is no special limitation on the solid-liquid separation, and any solid-liquid separation method well known to those skilled in the art, such as suction filtration, may be used. In the present disclosure, water used in the washing has a temperature of not greater than 5° C., preferably 0-5° C., and more preferably 1-4° C. In some embodiments, the water is deionized water. In the present disclosure, there is no special limitation on the number of times of washing, and as long as the solid product is washed to be neutral. In some embodiments, the drying is performed at a temperature of 20-40° C., preferably 25-35° C., and more preferably 40° C. In some embodiments, the drying is performed for 12-24 h, preferably 15-22 h, and more preferably 18-20 h. In some embodiments, the drying is vacuum drying.

The present disclosure provides use of the 3-nitro-1,2,4-triazole-5-one-d2 described in the technical solution or the 3-nitro-1,2,4-triazole-5-one-d2 prepared by any method described in the above technical solutions in national defense field. The 3-nitro-1,2,4-triazole-5-one-d2 prepared by the present disclosure has high crystal density, high thermal stability, high detonation performance and low friction sensitivity, and has great application value and important research significance in national defense field as an explosive (especially a low-sensitive high explosive) or as a raw material for preparing explosives.

In the following, the technical solutions of the present disclosure will be described clearly and completely in combination with the examples in the present disclosure. Obviously, the described examples are only a part of the embodiment of the present disclosure, not the whole embodiments. Based on the embodiments in the present disclosure, all other embodiments obtained by those skilled in the field without creative labor belong to the scope of the present disclosure.

### Example 1

11.7 g of deuterated semicarbazide hydrochloride was added into 11.3 g of a deuterated formic acid aqueous solution with a mass concentration of 85%, and mixed to be uniform to obtain a first mixed solution. The first mixed solution was transferred into a pressure-resistant container with a volume of 50 mL, and then the pressure-resistant container was sealed. The first mixed solution was stirred at a rate of 70 rpm and heated to 90° C., held at 90° C. for 2 h for reaction, then heated to 106° C., refluxed for 4 h under the protection of nitrogen, and then naturally cooled to ambient temperature. The cooled reaction solution was subjected to suction filtration to obtain a solid product. The solid product was dried in vacuum at 40° C. for 12 h, obtaining 9.1 g of a crude 1,2,4-triazole-5-one-d3 crystal.

A pressure-resistant container with a volume of 50 mL and containing 20 g of deionized water was placed in a high-precision medium-temperature circulating bath at 30° C. 8.7 g of the crude 1,2,4-triazole-5-one-d3 crystal was added into deionized water to obtain a mixture. The container was sealed. The mixture was heated to 80° C., stirred to dissolve the solid, then cooled to 20° C. at a cooling rate of 10° C./min, and then cooled to 5° C. at a cooling rate of 1° C./min. The cooled mixture was subjected to suction filtration to obtain a solid product. The solid product was washed by deionized water that has a temperature of not greater than 5° C. for 3 times, and then dried in vacuum at 40° C. for 12 h, obtaining 7.6 g 1,2,4-triazole-5-one-d3 crystal.

A pressure-resistant container with a volume of 100 mL and containing 7.6 g of 1,2,4-triazole-5-one-d3 crystal was placed in a high-precision medium-temperature circulating bath at −5° C. A deuterated nitric acid aqueous solution with a mass concentration of 90% was dropwise added into 1,2,4-triazole-5-one-d3 through a constant pressure dropping funnel at a rate of 1 mL/min within 33 min, and the system temperature was held not greater than 5° C. during the dropwise adding process. Then 9.9 g of phosphoric anhydride and 0.76 g of boron trichloride were sequentially added thereto to obtain a second mixed solution. The container was sealed. The second mixed solution was stirred at a rate of 400 rpm and heated to 60° C., held at 60° C. for 4 h for reaction, cooled to 10° C. at a cooling rate of 10° C./min, and then subjected to suction filtration to obtain a solid product. The solid product was washed with 5° C.-deionized water until the obtained filtrate was neutral, and then dried in vacuum at 30° C. for 20 h, obtaining 11.1 g of 3-nitro-1,2,4-triazole-5-one-d2 with a yield of 84.3% and a purity of 99.3%.

The 3-nitro-1,2,4-triazole-5-one-d2 has a crystal density of 1.933 g/cm³, a thermal decomposition temperature of 282.7° C., and a detonation velocity of 8832 m/s. The friction sensitivity of the 3-nitro-1,2,4-triazole-5-one-d2 was tested by a BAM tribometer, and the results show that the friction sensitivity of the sample was 366 N.

The non-deuterated 3-nitro-1,2,4-triazole-5-one has a detonation velocity of 8560 m/s, an impact sensitivity of greater than 280 cm, a friction sensitivity of greater than 353 N and a thermal decomposition temperature of 280° C. (see Ou Yuxiang, Explosives [M], Beijing institute of technology press, 2014).

It shows that compared with non-deuterated 3-nitro-1,2,4-triazole-5-one, the 3-nitro-1,2,4-triazole-5-one-d2 prepared by the present disclosure exhibits improved thermal stability and detonation performance, and reduced friction sensitivity.

### Example 2

18.8 g of deuterated semicarbazide hydrochloride was added into 36.1 g of a deuterated formic acid aqueous solution with a mass concentration of 85%, and mixed to be uniform to obtain a first mixed solution. The first mixed solution was transferred into a pressure-resistant container with a volume of 100 mL, and then the pressure-resistant container was sealed. The first mixed solution was stirred at a rate of 70 rpm and heated to 95° C., held at 95° C. for 2.5 h for reaction, then heated to 110° C., refluxed for 4 h under the protection of nitrogen, and then naturally cooled to ambient temperature. The cooled reaction solution was subjected to suction filtration to obtain a solid product. The solid product was dried in vacuum at 40° C. for 12 h, obtaining 14.4 g of a crude 1,2,4-triazole-5-one-d3 crystal.

A pressure-resistant container with a volume of 100 mL and containing 32 g of deionized water was placed in a high-precision medium-temperature circulating bath at 30° C. 14.4 g of the crude 1,2,4-triazole-5-one-d3 crystal was added into deionized water to obtain a mixture. The container was sealed. The mixture was heated to 80° C., stirred to dissolve the solid, then cooled to 20° C. at a cooling rate of 5° C./min, and then cooled to 5° C. at a cooling rate of 1° C./min. The cooled mixture was subjected to suction filtration to obtain a solid product. The solid product was washed by deionized water that has a temperature of not greater than 5° C. for 3 times, and then dried in vacuum at 40° C. for 12 h, obtaining 12.9 g 1,2,4-triazole-5-one-d3 crystal.

A pressure-resistant container with a volume of 200 mL and containing 12.9 g of 1,2,4-triazole-5-one-d3 crystal was placed in a high-precision medium-temperature circulating bath at −5° C. A deuterated nitric acid aqueous solution with a mass concentration of 90% was dropwise added into 1,2,4-triazole-5-one-d3 through a constant pressure dropping funnel at a rate of 1 mL/min within 63 min, and the system temperature was held not greater than 5° C. during the dropwise adding process. Then 17.1 g of phosphoric anhydride and 1.3 g of boron trichloride were sequentially added thereto to obtain a second mixed solution. The container was sealed. The second mixed solution was stirred at a rate of 400 rpm and heated to 60° C., held at 60° C. for 4 h for reaction, cooled to 10° C. at a cooling rate of 10° C./min, and then subjected to suction filtration to obtain a solid product. The solid product was washed with 5° C.-deionized water until pH of the obtained filtrate was neutral, and then dried in vacuum at 30° C. for 20 h, obtaining 18.9 g of 3-nitro-1,2,4-triazole-5-one-d2 with a yield of 89.4% and a purity of 99.6%.

The 3-nitro-1,2,4-triazole-5-one-d2 has a crystal density of 1.937 g/cm³, a thermal decomposition temperature of 283.2° C., and a detonation velocity is 8846 m/s, indicating better thermal stability and detonation performance than ordinary 3-nitro-1,2,4-triazole-5-one. The friction sensitivity of the 3-nitro-1,2,4-triazole-5-one-d2 was tested by a BAM tribometer, and the results show that the friction sensitivity of the sample was 373 N. Compared with non-deuterated 3-nitro-1,2,4-triazole-5-one, 3-nitro-1,2,4-triazole-5-one-d2 exhibits reduced friction sensitivity.

### Example 3

16.4 g of deuterated semicarbazide hydrochloride was added into 38.4 g of a deuterated formic acid aqueous solution with a mass concentration of 70%, and mixed to be uniform to obtain a first mixed solution. The first mixed solution was transferred into a pressure-resistant container with a volume of 100 mL, and then the pressure-resistant container was sealed. The first mixed solution was stirred at a rate of 70 rpm and heated to 95° C., held at 95° C. for 2.5 h for reaction, then heated to 110° C., refluxed for 4 h under the protection of nitrogen, and then naturally cooled to ambient temperature. The cooled reaction solution was subjected to suction filtration to obtain a solid product. The solid product was dried in vacuum at 40° C. for 12 h, obtaining 13.1 g of a crude 1,2,4-triazole-5-one-d3 crystal.

A pressure-resistant container with a volume of 100 mL and containing 29 g of methanol was placed in a high-precision medium-temperature circulating bath at 30° C. 13.1 g of the crude 1,2,4-triazole-5-one-d3 crystal was added into methanol to obtain a mixture. The container was sealed. The mixture was heated to 55° C., stirred to dissolve the solid, then cooled to 10° C. at a cooling rate of 5° C./min, and then cooled to 5° C. at a cooling rate of 1° C./min. The cooled mixture was subjected to suction filtration to obtain a solid product. The solid product was washed by deionized water that has a temperature of not greater than 5° C. for 3 times, and then dried in vacuum at 40° C. for 12 h, obtaining 9.9 g 1,2,4-triazole-5-one-d3 crystal.

A pressure-resistant container with a volume of 100 mL and containing 9.9 g of 1,2,4-triazole-5-one-d3 crystal was placed in a high-precision medium-temperature circulating bath at −5° C. A deuterated nitric acid aqueous solution with a mass concentration of 95% was dropwise added into 1,2,4-triazole-5-one-d3 through a constant pressure dropping funnel at a rate of 1 mL/min within 40 min, and the system temperature was held not greater than 5° C. during the dropwise adding process. Then 12.7 g of phosphoric anhydride and 1.0 g of boron trichloride were sequentially added thereto to obtain a second mixed solution. The container was sealed. The second mixed solution was stirred at a rate of 400 rpm and heated to 70° C., held at 70° C. for 5 h for reaction, cooled to 10° C. at a cooling rate of 10° C./min, and then subjected to suction filtration to obtain a solid product. The solid product was washed with 5° C.-deionized water until pH of the obtained filtrate was neutral, and then dried in vacuum at 30° C. for 20 h, obtaining 14.6 g of 3-nitro-1,2,4-triazole-5-one-d2 with a yield of 79.3% and a purity of 99.1%.

The 3-nitro-1,2,4-triazole-5-one-d2 has a crystal density of 1.930 g/cm³, a thermal decomposition temperature of 282.2° C., and a detonation velocity is 8828 m/s, indicating better thermal stability and detonation performance than ordinary 3-nitro-1,2,4-triazole-5-one. The friction sensitivity of the 3-nitro-1,2,4-triazole-5-one-d2 was tested by a BAM tribometer, and the results show that the friction sensitivity of the sample was 364 N. Compared with non-deuterated 3-nitro-1,2,4-triazole-5-one, 3-nitro-1,2,4-triazole-5-one-d2 exhibits reduced friction sensitivity.

### Example 4

7.1 g of deuterated semicarbazide hydrochloride was added into 16.0 g of a deuterated formic acid aqueous solution with a mass concentration of 90%, and mixed to be uniform to obtain a first mixed solution. The first mixed solution was transferred into a pressure-resistant container with a volume of 50 mL, then the pressure-resistant container was sealed. The first mixed solution was stirred at a rate of 70 rpm and heated to 95° C., held at 95° C. for 3 h for reaction, then heated to 110° C., refluxed for 6 h under the protection of nitrogen, and then naturally cooled to ambient temperature. The cooled reaction solution was subjected to suction filtration to obtain a solid product. The solid product was dried in vacuum at 40° C. for 12 h, obtaining 5.4 g of a crude 1,2,4-triazole-5-one-d3 crystal.

A pressure-resistant container with a volume of 50 mL and containing 16 g of ethanol was placed in a high-precision medium-temperature circulating bath at 30° C. 5.4 g of the crude 1,2,4-triazole-5-one-d3 crystal was added into ethanol to obtain a mixture. The container was sealed. The mixture was heated to 70° C., stirred to dissolve the solid, then cooled to 10° C. at a cooling rate of 5° C./min, and then cooled to 5° C. at a cooling rate of 1° C./min. The cooled mixture was subjected to suction filtration to obtain a solid product. The solid product was washed by deionized water that has a temperature of not greater than 5° C. for 3 times, and then dried in vacuum at 40° C. for 12 h, obtaining 4.9 g 1,2,4-triazole-5-one-d3 crystal.

A pressure-resistant container with a volume of 50 mL and containing 4.9 g of 1,2,4-triazole-5-one-d3 crystal was placed in a high-precision medium-temperature circulating bath at −5° C. A deuterated nitric acid aqueous solution with a mass concentration of 90% was dropwise added into 1,2,4-triazole-5-one-d3 through a constant pressure dropping funnel at a rate of 0.5 mL/min within 53 min, and the system temperature was held not greater than 5° C. during the dropwise adding process. Then 9.9 g of phosphoric anhydride and 1.0 g of aluminium trichloride were sequentially added thereto to obtain a second mixed solution. The container was sealed. The second mixed solution was stirred at a rate of 400 rpm and heated to 70° C., held at 70° C. for 4 h for reaction, cooled to 10° C. at a cooling rate of 10° C./min, and then subjected to suction filtration to obtain a solid product. The solid product was washed with 5° C.-deionized water until pH of the obtained filtrate was neutral, and then dried in vacuum at 30° C. for 20 h, obtaining 7.4 g of 3-nitro-1,2,4-triazole-5-one-d2 with a yield of 92.8% and a purity of 99.7%.

The 3-nitro-1,2,4-triazole-5-one-d2 has a crystal density of 1.939 g/cm³, a thermal decomposition temperature of 283.4° C., and a detonation velocity is 8848 m/s, indicating better thermal stability and detonation performance than ordinary 3-nitro-1,2,4-triazole-5-one. The friction sensitivity of the 3-nitro-1,2,4-triazole-5-one-d2 was tested by a BAM tribometer, and the results show that the friction sensitivity of the sample was 377 N. Compared with non-deuterated 3-nitro-1,2,4-triazole-5-one, 3-nitro-1,2,4-triazole-5-one-d2 exhibits reduced friction sensitivity.

### Example 5

35.3 g of deuterated semicarbazide hydrochloride was added into 53.3 g of a deuterated formic acid deionized water solution with a mass concentration of 90%, and mixed to be uniform to obtain a first mixed solution. The first mixed solution was transferred into a pressure-resistant container with a volume of 150 mL, and then the pressure-resistant container was sealed. The first mixed solution was stirred at a rate of 70 rpm and heated to 95° C., held at 95° C. for 3 h for reaction, then heated to 120° C., refluxed for 5 h under the protection of nitrogen, and then naturally cooled to ambient temperature. The cooled reaction solution was subjected to suction filtration to obtain a solid product. The solid product was dried in vacuum at 40° C. for 12 h, obtaining 27.7 g of a crude 1,2,4-triazole-5-one-d3 crystal.

A pressure-resistant container with a volume of 200 mL and containing 70 g of ethylene glycol was placed in a high-precision medium-temperature circulating bath at 30° C. 27.7 g of the crude 1,2,4-triazole-5-one-d3 crystal was added into ethylene glycol to obtain a mixture. The container was sealed. The mixture was heated to 120° C., stirred to dissolve the solid, then cooled to 10° C. at a cooling rate of 10° C./min, and then cooled to 5° C. at a cooling rate of 1° C./min. The cooled mixture was subjected to suction filtration to obtain a solid product. The solid product was washed by deionized water that has a temperature of not greater than 5° C. for 3 times, and then dried in vacuum at 40° C. for 12 h, obtaining 22.4 g 1,2,4-triazole-5-one-d3 crystal.

A pressure-resistant container with a volume of 200 mL and containing 22.4 g of 1,2,4-triazole-5-one-d3 crystal was placed in a high-precision medium-temperature circulating bath at −5° C. A deuterated nitric acid aqueous solution with a mass concentration of 92% was dropwise added into 1,2,4-triazole-5-one-d3 through a constant pressure dropping funnel at a rate of 2 mL/min within 53 min, and the system temperature was held not greater than 5° C. during the dropwise adding process. Then 28.4 g of phosphoric anhydride and 4.4 g of ferric trichloride were sequentially added thereto to obtain a second mixed solution. The container was sealed. The second mixed solution was stirred at a rate of 400 rpm and heated to 70° C., held at 70° C. for 4 h for reaction, cooled to 10° C. at a cooling rate of 10° C./min, and then subjected to suction filtration to obtain a solid product. The solid product was washed with 5° C.-deionized water until pH of the obtained filtrate was neutral, and then dried in vacuum at 30° C. for 20 h, obtaining 33.1 g of 3-nitro-1,2,4-triazole-5-one-d2 with a yield of 83.3% and a purity of 99.6%.

The 3-nitro-1,2,4-triazole-5-one-d2 has a crystal density of 1.937 g/cm³, a thermal decomposition temperature of 283.1° C., and a detonation velocity is 8846 m/s, indicating better thermal stability and detonation performance than ordinary 3-nitro-1,2,4-triazole-5-one. The friction sensitivity of the 3-nitro-1,2,4-triazole-5-one-d2 was tested by a BAM tribometer, and the results show that the friction sensitivity of the sample was 374 N. Compared with non-deuterated 3-nitro-1,2,4-triazole-5-one, 3-nitro-1,2,4-triazole-5-one-d2 exhibits reduced friction sensitivity.

The above is only preferred embodiments of the present disclosure, and it should be pointed out that those skilled in the art could make several improvements and modifications without departing from the principle of the present disclosure, and these improvements and modifications shall also fall within the scope of the present disclosure.

What is claimed is:

1. 3-nitro-1,2,4-triazole-5-one-d2, having a structure represented by formula I:

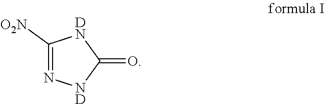

2. A method for preparing the 3-nitro-1,2,4-triazole-5-one-d2 of claim 1, comprising the following steps:
mixing deuterated semicarbazide hydrochloride and a deuterated formic acid aqueous solution to obtain a first mixed solution, subjecting the first mixed solution to condensation reaction to obtain a crude 1,2,4-triazole-5-one-d3, and subjecting the crude 1,2,4-triazole-5-one-d3 to recrystallization to obtain 1,2,4-triazole-5-one-d3, and
mixing the 1,2,4-triazole-5-one-d3, a deuterated nitric acid aqueous solution, phosphoric anhydride and a chloride at a temperature of not greater than 5° C. to obtain a second mixed solution, and subjecting the second mixed solution to nitration reaction to obtain the 3-nitro-1,2,4-triazole-5-one-d2.

3. The method of claim 2, wherein the deuterated formic acid aqueous solution has a mass concentration of 60-90%, and
a molar ratio of the deuterated semicarbazide hydrochloride to deuterated formic acid in the deuterated formic acid aqueous solution is in a range of 1:(2-6).

4. The method of claim 3, wherein the condensation reaction comprises first condensation reaction and second condensation reaction which are sequentially performed, and
the first condensation reaction is performed at a temperature of 90-95° C. for 2-3 h, and the second condensation reaction is performed at a temperature of 100-120° C. for 4-6 h in a protective atmosphere.

5. The method of claim 2, wherein the condensation reaction comprises first condensation reaction and second condensation reaction which are sequentially performed, and
the first condensation reaction is performed at a temperature of 90-95° C. for 2-3 h, and the second condensation reaction is performed at a temperature of 100-120° C. for 4-6 h in a protective atmosphere.

6. The method of claim 2, wherein a recrystallization solvent used in the recrystallization comprises one or more selected from the group consisting of water and an alcohol, and the alcohol comprises one or more selected from the group consisting of methanol, ethanol, ethylene glycol, propanol and glycerol, and
a mass ratio of the crude 1,2,4-triazole-5-one-d3 to the recrystallization solvent is in a range of 1:(2-5).

7. The method of claim 6, wherein the recrystallization comprises thermal dissolution, first cooling crystallization and second cooling crystallization which are sequentially performed,
the thermal dissolution is performed at a temperature of 50-100° C.,
the first cooling crystallization has a final temperature of 10-20° C., and is performed at a cooling rate of 1-10° C./min, and
the second cooling crystallization has a final temperature of not greater than 5° C., and is performed at a cooling rate of 0.5-1° C./min.

8. The method of claim 2, wherein the recrystallization comprises thermal dissolution, first cooling crystallization and second cooling crystallization which are sequentially performed,
the thermal dissolution is performed at a temperature of 50-100° C.,
the first cooling crystallization has a final temperature of 10-20° C., and is performed at a cooling rate of 1-10° C./min, and
the second cooling crystallization has a final temperature of not greater than 5° C., and is performed at a cooling rate of 0.5-1° C./min.

9. The method of claim 2, wherein mixing the 1,2,4-triazole-5-one-d3, a deuterated nitric acid aqueous solution, phosphoric anhydride and a chloride is performed as follows: dropwise adding the deuterated nitric acid aqueous solution into the 1,2,4-triazole-5-one-d3, then sequentially adding phosphoric anhydride and the chloride, and mixing,
wherein the 1,2,4-triazole-5-one-d3 has a temperature of −5° C. to 0° C., and
the deuterated nitric acid aqueous solution is dropwise added at a rate of 0.5-3 mL/min.

10. The method of claim 9, wherein the deuterated nitric acid aqueous solution has a mass concentration of 90-95%, and
a molar ratio of the 1,2,4-triazole-5-one-d3 to deuterated nitric acid in the deuterated nitric acid aqueous solution is in a range of 1:(8-12);
a molar ratio of phosphoric anhydride to deuterated nitric acid in the deuterated nitric acid aqueous solution is in a range of 1:(5-10);
a mass ratio of the 1,2,4-triazole-5-one-d3 to the chloride is in a range of (5-10): 1, and
the chloride comprises one or more selected from the group consisting of boron trichloride, aluminum trichloride, silicon tetrachloride and ferric trichloride.

11. The method of claim 9, wherein the nitration reaction is performed at a temperature of 60-80° C. for 4-8 h.

12. The method of claim 2, wherein the deuterated nitric acid aqueous solution has a mass concentration of 90-95%, and
a molar ratio of the 1,2,4-triazole-5-one-d3 to deuterated nitric acid in the deuterated nitric acid aqueous solution is in a range of 1:(8-12);
a molar ratio of phosphoric anhydride to deuterated nitric acid in the deuterated nitric acid aqueous solution is in a range of 1:(5-10);
a mass ratio of the 1,2,4-triazole-5-one-d3 to the chloride is in a range of (5-10): 1, and
the chloride comprises one or more selected from the group consisting of boron trichloride, aluminum trichloride, silicon tetrachloride and ferric trichloride.

13. The method of claim 2, wherein the nitration reaction is performed at a temperature of 60-80° C. for 4-8 h.

14. An explosive, comprising the 3-nitro-1,2,4-triazole-5-one-d2 of claim 1.
